(19) United States
(12) Patent Application Publication
(10) Pub. No.: US 2026/0083437 A1
(43) Pub. Date: Mar. 26, 2026

(54) ULTRASOUND IMAGE PROCESSING APPARATUS AND ULTRASOUND IMAGE PROCESSING PROGRAM

(71) Applicant: FUJIFILM Corporation, Tokyo (JP)

(72) Inventors: Kenta INOUE, Tokyo (JP); Takehiro TSUJITA, Tokyo (JP); Tomofumi NISHIURA, Tokyo (JP); Kosei TAKEMOTO, Tokyo (JP)

(73) Assignee: FUJIFILM Corporation, Tokyo (JP)

(21) Appl. No.: 19/331,857

(22) Filed: Sep. 17, 2025

(30) Foreign Application Priority Data
Sep. 25, 2024 (JP) 2024-166474

(51) Int. Cl. A61B 8/00; A61B 8/08

(52) U.S. Cl. CPC A61B 8/463; A61B 8/085; A61B 8/565

(57) ABSTRACT

An ultrasound diagnostic apparatus includes a processor. The processor is configured to acquire an evaluation value for each item of ultrasound image data sequentially acquired based on transmission and reception of ultrasound, sequentially update and display an evaluation graph showing the evaluation value in a case where ultrasound images based on the ultrasound image data sequentially acquired are displayed sequentially, and display a memory map for the ultrasound image data sequentially acquired based on the transmission and the reception of the ultrasound. The memory map indicates a storage location in a memory where each item of the ultrasound image data is stored.

### CROSS REFERENCE TO RELATED APPLICATION

This application claims priority to Japanese Patent Application No. 2024-166474 filed on Sep. 25, 2024, which is incorporated herein by reference in its entirety including the specification, claims, drawings, and abstract.

### BACKGROUND OF THE INVENTION

### 1. Field of the Invention

The present disclosure relates to an ultrasound image processing apparatus and an ultrasound image processing program, and particularly to a technique for displaying an evaluation value for ultrasound image data.

### 2. Description of the Related Art

An ultrasound diagnostic apparatus has been widely used as an apparatus for observing a subject. The ultrasound diagnostic apparatus sequentially generates ultrasound image data of the subject over time by transmitting and receiving ultrasound, and sequentially displays images based on the ultrasound image data on a display.

In general, the ultrasound diagnostic apparatus comprises a cine memory that stores the ultrasound image data sequentially generated over time. The images (ultrasound images) based on the ultrasound image data are sequentially displayed on the display, and the ultrasound image data is stored in the cine memory. As a result, the cine memory stores a series of ultrasound image data generated over a certain period in the past. The ultrasound diagnostic apparatus designates the ultrasound image data stored in the cine memory based on a user's operation, and displays an image based on a designated frame on the display.

In the ultrasound diagnostic apparatus, an operation of sequentially acquiring the ultrasound image data over time while transporting an ultrasound probe may be performed. This transport operation causes a series of ultrasound image data for a region in the subject to be stored in the cine memory.

As processing of displaying an evaluation value for the series of ultrasound image data stored in the cine memory, there is scrolling graph display. In this display, the ultrasound image data is sequentially read out from the cine memory, and, as the ultrasound images are sequentially displayed, figures indicating the evaluation values are arranged and displayed in a horizontal direction in time-series order. For example, bars as figures that extend in a vertical direction with lengths corresponding to the evaluation values are arranged and displayed in time-series order from left to right. As a result, each evaluation value acquired for the region in the subject is visualized on a graph.

JP2023-90023A discloses, as a graph displayed by the scrolling graph display, a graph (second reference image) showing a change in time of a lesion part probability. Here, the lesion part probability refers to a value indicating a degree of the likelihood that a specific region appearing in the ultrasound image is a lesion.

### SUMMARY OF THE INVENTION

In the scrolling graph display, the graph may be displayed in an address range narrower than an address range corresponding to the ultrasound image data stored in the cine memory. In this case, it may be difficult for the user to ascertain which portion of the entire address range corresponding to the ultrasound image data stored in the cine memory corresponds to the address range subjected to the scrolling graph display. As a result, it may be difficult for the user to ascertain which region of the subject is represented by the ultrasound image subjected to the scrolling graph display.

An object of the present disclosure is to facilitate diagnosis based on display of an evaluation value for ultrasound image data.

An ultrasound image processing apparatus according to the present disclosure comprises: a processor configured to acquire an evaluation value for each item of ultrasound image data sequentially acquired based on transmission and reception of ultrasound, sequentially update and display an evaluation graph showing the evaluation value in a case where ultrasound images based on the ultrasound image data sequentially acquired are displayed sequentially, and display a memory map for the ultrasound image data sequentially acquired based on the transmission and the reception of the ultrasound, in which the memory map indicates a storage location in a memory where each item of the ultrasound image data is stored.

In one embodiment, the memory map is a map that indicates the storage location in the memory with a frame number, the evaluation graph is a graph in which the evaluation value is displayed in association with a frame number axis, and the processor is configured to display, on the memory map, an evaluation range on the frame number axis that is targeted for display in the evaluation graph in a form of a figure.

In one embodiment, the processor is configured to determine a range of frame numbers of ultrasound image data whose evaluation value satisfies a lesion condition among the ultrasound image data of a plurality of frames stored in the memory, and to display the determined range on the memory map.

In one embodiment, the processor is configured to determine index ultrasound image data whose evaluation value satisfies an index condition among the ultrasound image data of a plurality of frames stored in the memory, and display an index frame point indicating a storage location in the memory where the index ultrasound image data is stored, together with the memory map.

In one embodiment, the processor is configured to display a memory map operation figure obtained by superimposing the memory map on an operation figure for designating any of the ultrasound image data of a plurality of frames stored in the memory, and to display an image based on the ultrasound image data designated by the operation figure.

In one embodiment, the evaluation graph is a graph in which elongated figures that extend in a first axis direction with lengths corresponding to the evaluation values are displayed in a sequential arrangement in a second axis direction as the ultrasound images based on the ultrasound image data are sequentially displayed.

In one embodiment, the evaluation value is a value indicating a degree to which the ultrasound image indicated by the ultrasound image data includes an atypical region.

In one embodiment, the processor is configured to arrange and display a plurality of the evaluation graphs indicating a plurality of different types of the evaluation values.

In one embodiment, the different types of the evaluation values are at least two values of a tumor category, a degree of ease of recognition of a tumor boundary, a tumor aspect ratio, and a level of reflected ultrasound.

In addition, an ultrasound image processing program according to the present disclosure causes a processor to execute a process comprising: acquiring an evaluation value for each item of ultrasound image data sequentially acquired based on transmission and reception of ultrasound; sequentially updating and displaying an evaluation graph showing the evaluation value in a case where ultrasound images based on the ultrasound image data sequentially acquired are displayed sequentially; and displaying a memory map for the ultrasound image data sequentially acquired based on the transmission and the reception of the ultrasound.

According to the present disclosure, it is possible to facilitate diagnosis based on display of an evaluation value for ultrasound image data.

### BRIEF DESCRIPTION OF THE DRAWINGS

FIG. 1 is a diagram showing a configuration of an ultrasound diagnostic apparatus.

FIG. 2 is a diagram showing processing of acquiring B-mode image data.

FIG. 3 is a diagram showing an example of an image displayed on a display.

FIG. 4 is a diagram showing an example of an image displayed on the display.

FIG. 5 is a diagram showing a screening graph in an enlarged manner.

FIGS. 6A to 6E are diagrams showing a method for dynamically displaying a screening graph.

FIG. 7 is a diagram showing an example of a memory map seek bar.

FIG. 8 is a diagram showing an image in which a screening graph is displayed for each of a plurality of different types of evaluation values.

FIG. 9 is a diagram showing an example of a screening graph.

FIG. 10 is a diagram showing an image in which screening graphs having different numbers of frames are compared.

FIG. 11 is a diagram showing B-mode image data and a lesion candidate region.

FIG. 12 is a diagram showing an example of a memory map seek bar.

### DESCRIPTION OF THE PREFERRED EMBODIMENTS

An embodiment of the present disclosure will be described with reference to the drawings. The same components shown in a plurality of drawings are denoted by the same reference numerals to simplify the description thereof.

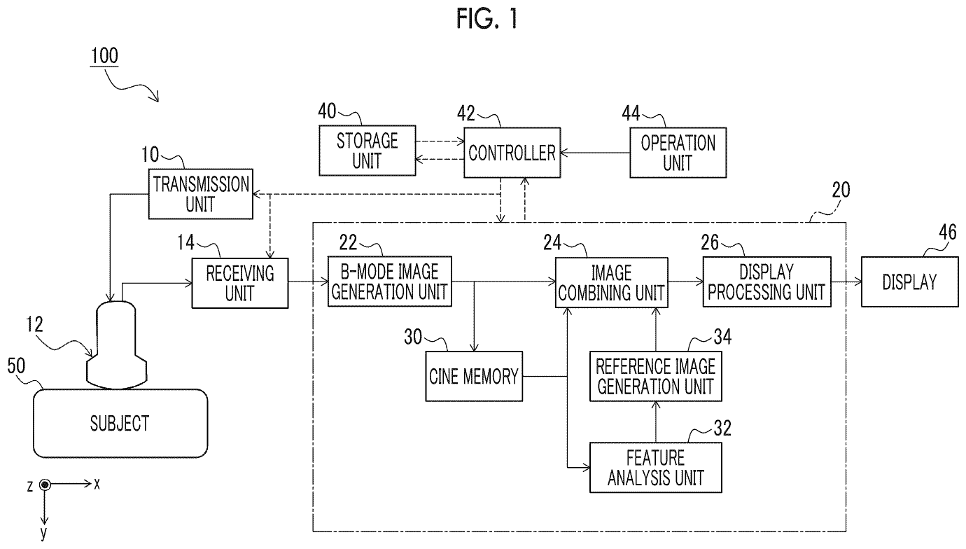

FIG. 1 shows a configuration of an ultrasound diagnostic apparatus 100 according to an embodiment of the present disclosure. The ultrasound diagnostic apparatus 100 comprises a transmission unit 10, an ultrasound probe 12, a receiving unit 14, an information processing unit 20, a controller 42, an operation unit 44, and a display 46 (display unit).

The operation unit 44 may comprise a button, a lever, a keyboard, a mouse, and the like. The operation unit 44 may be a touch panel provided on the display 46. A storage unit 40 shown in FIG. 1 together with the ultrasound diagnostic apparatus 100 may be a storage (storage medium) such as a hard disk implemented in the ultrasound diagnostic apparatus 100. In addition, the storage unit 40 may be a memory (storage medium) of a computer on a local area network or a memory of a computer on an electric communication line such as the Internet.

The information processing unit 20 comprises a B-mode image generation unit 22, an image combining unit 24, a display processing unit 26, a cine memory 30, a feature analysis unit 32, and a reference image generation unit 34. The information processing unit 20 and the controller 42 may be configured by, for example, one or a plurality of computers that execute a program stored in the storage unit 40.

That is, in the ultrasound diagnostic apparatus 100 according to the present embodiment, each process is executed by any computer. In addition, any computer may execute these processes using a processor as hardware, a program as software, or a combination thereof. In that case, the processor is configured to execute various processes in the present embodiment in cooperation with the program, and can function as each unit or each means in the present embodiment. In addition, the order in which the processes are executed by the processor is not limited to the order described above and may be changed as appropriate. Any computer may be a general-purpose computer, a computer for a specific use, a workstation, or another system capable of executing each process.

The processor may be configured by one or more pieces of hardware, and the type of hardware is not limited. For example, the processor may be configured by a programmable logic device such as a central processing unit (CPU), a micro processing unit (MPU), or a field programmable gate array (FPGA), a dedicated circuit for executing specific processing such as an application specific integrated circuit (ASIC), or hardware such as a graphics processing unit (GPU) or a neural processing unit (NPU). In addition, the types of hardware may be a combination of different types of hardware. In a case where a plurality of pieces of hardware are configured to execute one or a plurality of processes of a certain processor, the plurality of pieces of hardware may be present in devices physically separated from each other, or may be present in the same device. In addition, in any of the embodiments, the order of each processing executed by the processor is not limited to the above order and may be changed as appropriate. The hardware is configured by an electric circuit (circuitry) in which circuit elements such as semiconductor elements are combined.

Further, the program may be software such as firmware or a microcode. In addition, the program may be, for example, a program module group, and each function thereof may be realized by a processor configured to execute each function. The program may be a program code or a plurality of code segments stored in one or a plurality of non-transitory computer-readable media (for example, a storage medium or other storage). The program may be divided and stored in a plurality of non-transitory computer-readable media present in devices physically separated from each other. The program code or the code segment may represent any combination of a procedure, a function, a subprogram, a routine, a subroutine, a module, a software package, a class, an instruction, a data structure, or a program statement. The program code or the code segment may be connected to another code segment or a hardware circuit by transmitting and receiving information, data, an argument, a parameter, or memory contents.

The information processing unit 20 executes a program to configure each component (the B-mode image generation unit 22, the image combining unit 24, the display processing unit 26, the cine memory 30, the feature analysis unit 32, and the reference image generation unit 34) and to operate as an ultrasound image processing apparatus. The controller 42 may acquire information generated by each component in the information processing unit 20. In addition, the controller 42 may control the transmission unit 10, the receiving unit 14, and the information processing unit 20 in response to a user's operation of the operation unit 44.

The ultrasound probe 12 comprises a plurality of ultrasound transducers arranged along a contact surface facing a positive y-axis direction side. In the present embodiment, a plurality of ultrasound transducers are arranged in one or a plurality of columns in a major axis direction (x-axis direction). The ultrasound transducers are arranged in one or a plurality of rows in a minor axis direction (z-axis direction).

The transmission unit 10 outputs a transmission signal to each ultrasound transducer, and each ultrasound transducer generates an ultrasound wave in response to the transmission signal output from the transmission unit 10 to itself. The transmission unit 10 adjusts a delay time of the transmission signal output to each ultrasound transducer such that the ultrasound waves emitted from the respective ultrasound transducers constructively interfere in a specific transmission beam direction. As a result, a transmission beam is formed in the transmission beam direction. The transmission unit 10 adjusts the delay time of the transmission signal output to each ultrasound transducer to change a direction of the transmission beam, and scans an observation surface of a subject 50 with the transmission beam.

The respective ultrasound transducers receive reflected ultrasound generated by being reflected within the subject 50, convert the reflected ultrasound into a reception signal, which is an electric signal, and output the reception signal to the receiving unit 14. The receiving unit 14 adjusts the delay time of each reception signal output from each ultrasound transducer and performs synthesis processing, such as phasing addition, such that a plurality of reception signals based on ultrasound waves arriving from a direction in which the transmission beam is directed constructively interfere with each other, thereby generating reception beam data. The reception beam data is data based on the ultrasound wave reflected in the direction in which the transmission beam is directed. As a result, directivity is created for the ultrasound wave arriving from the direction in which the transmission beam is directed, and a reception beam is formed in the direction in which the transmission beam is directed. In the following description, the transmission beam and the reception beam are collectively referred to as transmission and reception beams.

The receiving unit 14 generates reception beam data corresponding to each direction of the transmission and reception beams scanned over the subject 50, and outputs the reception beam data to the B-mode image generation unit 22. The B-mode image generation unit 22 repeatedly scans the observation surface of the subject 50 with the transmission and reception beams over time, and sequentially generates B-mode image data as the ultrasound image data over time. The B-mode image generation unit 22 outputs the B-mode image data to the image combining unit 24 and the cine memory 30. The B-mode image data is associated with a frame number as information for identifying the B-mode image data, and then stored in the cine memory 30.

The ultrasound diagnostic apparatus 100 according to the present embodiment has operation modes of a real-time display mode and a freeze mode. The real-time display mode is an operation mode in which the B-mode image is displayed in real time on the display 46 as the display unit. That is, the real-time display mode is an operation mode in which the B-mode images are sequentially displayed on the display 46 over time based on the B-mode image data sequentially generated over time.

The image combining unit 24 performs image quality adjustment processing, such as brightness adjustment, contrast adjustment, and contour highlighting, on the B-mode image data, and outputs the B-mode image data after the image quality adjustment processing to the display processing unit 26. The display processing unit 26 sequentially converts the B-mode image data output from the image combining unit 24 into a video signal over time, and outputs the video signal to the display 46. The display 46 displays a real-time B-mode image based on the video signal.

In the real-time display mode, the B-mode image data output from the B-mode image generation unit 22 is stored in the cine memory 30 for a certain number of frames in the past, including the latest B-mode image data. As the latest B-mode image data is generated, the B-mode image data stored in the cine memory 30 first may be deleted from the cine memory 30.

The freeze mode is an operation mode in which images based on the B-mode image data of a plurality of frames stored in the cine memory 30 are displayed on the display 46. In the freeze mode, playback display may be performed in which the B-mode image data of a plurality of frames stored in the cine memory 30 are designated and displayed one frame at a time in time-series order. In addition, rewind playback display may be performed in which the B-mode image data of a plurality of frames stored in the cine memory 30 are designated and displayed one frame at a time in reverse time-series order. Further, any one of the B-mode image data of a plurality of frames stored in the cine memory 30 may be designated, and a still image based on the designated B-mode image data of one frame may be displayed.

Immediately before the operation mode is switched from the real-time display mode to the freeze mode, for example, the user may linearly move the observation surface by manually transporting the ultrasound probe 12 linearly in the minor axis direction (z-axis direction).

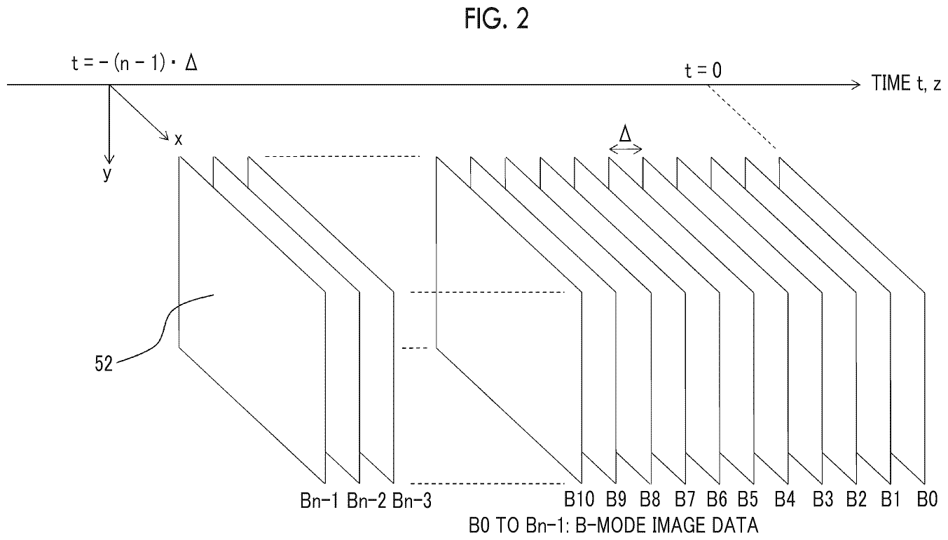

FIG. 2 conceptually shows processing of acquiring the B-mode image data in a case where the ultrasound probe 12 is linearly transported over the subject 50 in the minor axis direction at a constant speed during the operation in the real-time display mode. The ultrasound probe 12 is transported while an observation surface 52 scanned with the transmission and reception beams is aligned in a direction along an xy plane. An axis extending in a horizontal direction is a time axis (t-axis) or a z-axis, and the xy plane is defined perpendicular to the time axis. A plane parallel to the xy plane is scanned with an ultrasonic beam, so that the B-mode image indicated by each B-mode image data spreads parallel to the xy plane, and a plurality of B-mode images are linked together along the time axis.

In this process, the ultrasound diagnostic apparatus 100 operates in the real-time display mode before time point t=0, and an operation of setting the operation mode to the freeze mode is performed at time point t=0. During time point t=0 back to time point t=−(n−1)·Δ, the B-mode image data is acquired at every time interval A and stored in the cine memory 30. Each of rectangles B0 to Bn−1 arranged in parallel with the xy plane at the time interval A on the time axis conceptually indicates the B-mode image indicated by the B-mode image data.

In the freeze mode, the playback display of the B-mode image data of a plurality of frames stored in the cine memory 30 may be performed by the user's operation on the operation unit 44.

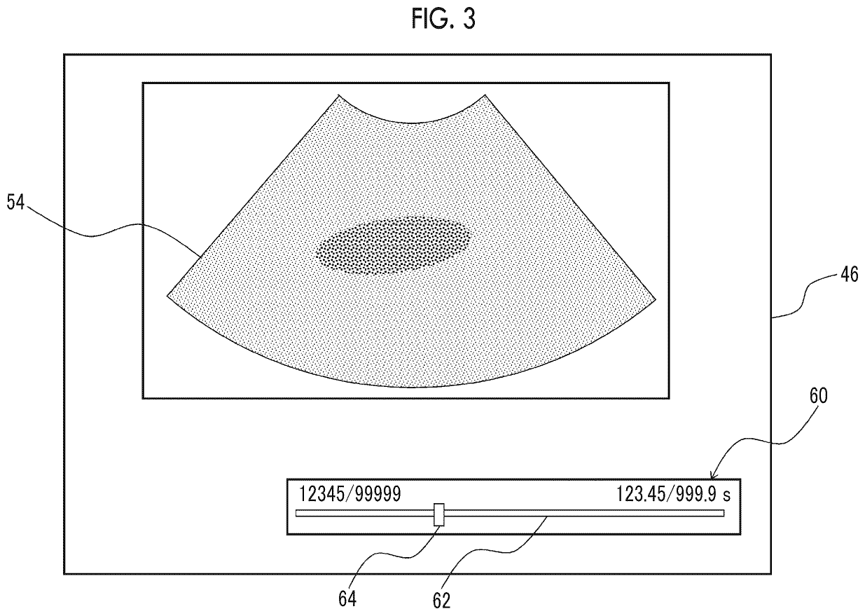

FIG. 3 shows an example of an image displayed on the display 46 in the operation of the freeze mode. In the image displayed on the display 46, a seek bar 60 for designating a frame number and a B-mode image 54 corresponding to the frame number designated by the seek bar 60 are depicted. The seek bar 60 is an operation figure for designating any of the ultrasound image data of a plurality of frames stored in the cine memory 30. The seek bar 60 includes a pointer 64 and a band-shaped FIG. 62 that extends to the left and right to designate the frame number. A horizontal position on the band-shaped FIG. 62 indicates a virtual storage location in the cine memory 30. The storage location is represented by a frame number.

In the present embodiment, the cine memory 30 stores B-mode images of 100,000 frames, with frame numbers ranging from 0 to 99999. A left end of the band-shaped FIG. 62 indicates the minimum value 0 of the frame number, and a right end indicates the maximum value 99999 of the frame number. A position of the pointer 64 on the band-shaped FIG. 62 indicates the frame number, and the frame number to be designated is changed by moving the pointer 64 left and right with a cursor or the like.

In the seek bar 60 shown in FIG. 3, a notation of “12345/99999” on a left side indicates that a B-mode image corresponding to the frame number 12345 among the frame numbers 0 to 99999 is displayed. In addition, a notation “123.45/999.9s” on a right side is a frame number converted into time (seconds). In the example shown in FIG. 3, the frame number 12345 is designated by the pointer 64, and the B-mode image 54 corresponding to the frame number 12345 is displayed.

By moving the pointer 64 from left to right, the B-mode images 54 are displayed one frame at a time from the past toward time point t=0, and the playback display is performed. By moving the pointer 64 from right to left, the B-mode images are displayed one frame at a time toward the past, and the rewind playback display is performed.

The process executed by the ultrasound diagnostic apparatus 100 for such display will be described below with reference to FIG. 1. The reference image generation unit 34 generates seek bar image data for displaying the seek bar 60, and outputs the seek bar image data to the image combining unit 24. The image combining unit 24 outputs display image data including at least the seek bar 60 to the display processing unit 26. The display processing unit 26 generates a video signal based on the display image data, and outputs the video signal to the display 46. The display 46 displays a display image including the seek bar 60 based on the video signal.

The user who refers to the seek bar 60 designates a frame number by operating the pointer 64 in the seek bar 60. The image combining unit 24 reads B-mode image data corresponding to the designated frame number from the cine memory 30. The image combining unit 24 generates display image data indicating the B-mode image and the seek bar 60 based on the B-mode image data and the seek bar image data output from the reference image generation unit 34, and outputs the display image data to the display processing unit 26. The display processing unit 26 generates a video signal based on the display image data, and outputs the video signal to the display 46. The display 46 displays a display image including the B-mode image and the seek bar 60 based on the video signal.

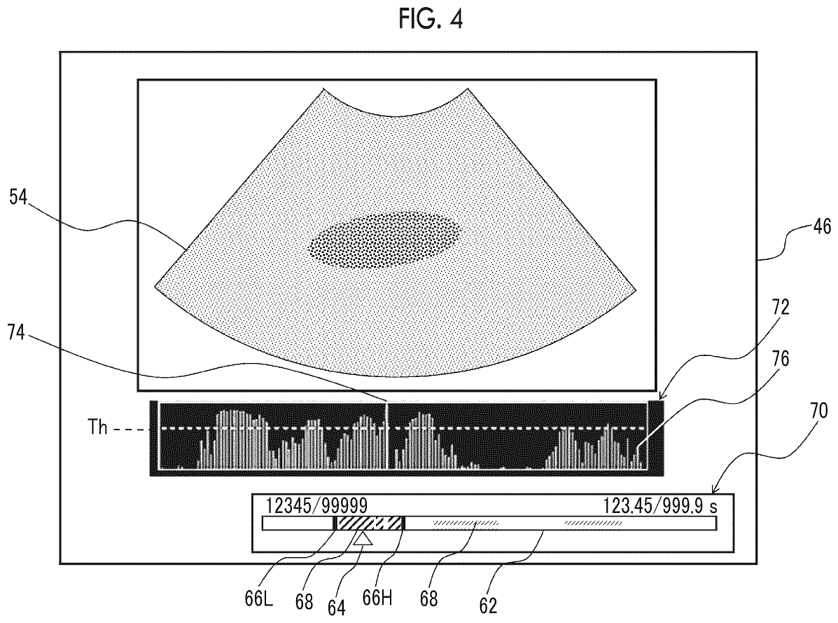

In the operation of the freeze mode, instead of the image shown in FIG. 3, the B-mode image 54, a screening graph 72, and a memory map seek bar 70 may be displayed on the display 46, as shown in FIG. 4. The screening graph 72 is an evaluation graph in which a frame number is taken on a horizontal axis and an evaluation value of the B-mode image is taken on a vertical axis, with the evaluation value being represented by a bar graph on the frame number axis. That is, the screening graph 72 is an evaluation graph in which the evaluation value is displayed in association with the frame number axis. The evaluation value indicates a degree to which the image indicated by the B-mode image data includes a region indicating a lesion as an atypical region.

On the screening graph 72, the above-described scrolling graph display may be performed. That is, the screening graph 72 as the evaluation graph may be a graph in which elongated figures that extend in a vertical axis direction (first axis direction) with lengths corresponding to the evaluation values are displayed in a sequential arrangement in a horizontal axis direction (second axis direction) as the images based on the B-mode image data are sequentially displayed.

On the screening graph 72 shown in FIG. 4, rod-like elements 76 as elongated figures indicating the evaluation values are arranged in time-series order in correspondence with the B-mode images of the plurality of frames that are virtually arranged on the frame number axis. The rod-like element 76 is a figure extending in the vertical axis direction.

The memory map seek bar 70 is a memory map operation figure obtained by superimposing a memory map on the seek bar 60 (operation figure) described above. The memory map is a diagram conceptually showing a storage location of the B-mode image data in the cine memory 30 with a frame number. The memory map in the present embodiment is displayed on the display 46 as the memory map seek bar 70 in a manner of being superimposed on the seek bar 60 (operation figure). Since specific B-mode image data is stored in or read out from the cine memory 30 by designating a frame number, conceptually, it may be considered that the storage location of the B-mode image data is represented by the frame number. In the present embodiment, a range of the frame numbers targeted for display on the screening graph 72 is shown on the memory map seek bar 70.

In the memory map seek bar 70 shown in FIG. 4, the pointer 64 for designating the frame number of the B-mode image to be displayed on the display 46 is indicated by a triangle whose apex is directed upward. Details of the functions of the memory map seek bar 70 will be described below.

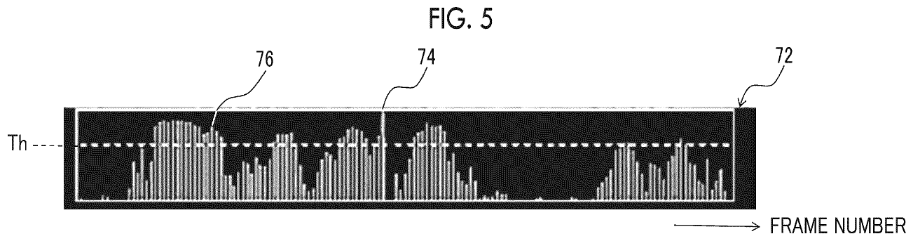

FIG. 5 shows the screening graph 72 in an enlarged manner. In the present embodiment, the larger the evaluation value, the greater the degree to which the lesion is shown on the B-mode image. A broken line indicating a threshold value Th is shown as a criterion for determining, based on the evaluation value, the likelihood that a tumor appearing in the B-mode image is malignant. In this example, a malignant tumor may appear in the B-mode image corresponding to the frame number in which the evaluation value exceeds the threshold value Th.

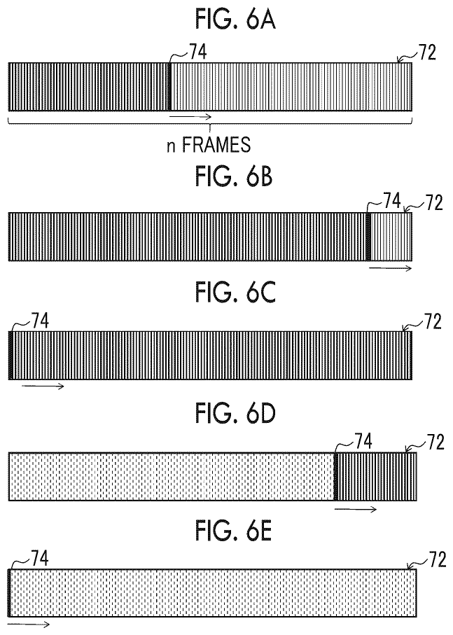

FIGS. 6A to 6E schematically show a method for dynamically displaying the screening graph 72. In this display method, in a case where images based on the B-mode image data sequentially acquired over time are sequentially displayed, the screening graph 72 is sequentially updated and displayed. On the screening graph 72, evaluation values for n frames are displayed. In a case where the B-mode image data of N frames is stored in the cine memory 30, evaluation values for n frames out of the N frames are shown on the screening graph 72. Here, N and n are positive integers, and N is larger than n.

A linear evaluation value indicator 74 that extends in the vertical direction is shown on the screening graph 72. The rod-like element 76 on which the evaluation value indicator 74 overlaps may be seen through the evaluation value indicator 74. The evaluation value indicated at a position of the evaluation value indicator 74 indicates the evaluation value of the B-mode image 54 displayed on the display 46. Since the B-mode image 54 displayed on the display 46 is designated by the pointer 64 in the memory map seek bar 70, the evaluation value indicator 74 indicates the evaluation value of the B-mode image 54 designated by the pointer 64. The evaluation value indicator 74 indicates a j-th evaluation value counted from the left, with the leftmost value indexed as 0, and this evaluation value corresponds to the evaluation value of the B-mode image 54 displayed on the display 46. Here, j is an integer of 0 or more.

For example, as shown in FIGS. 6A and 6B, in a case where the playback display is performed by sliding the pointer 64 in the memory map seek bar 70 from a position of the frame number 0 in a right direction, the evaluation value indicator 74 on the screening graph 72 moves in the right direction until the frame number indicated by the pointer 64 reaches n−1. The respective evaluation values corresponding to the B-mode image data for the frame numbers 0 to j are shown on the left side of the evaluation value indicator 74, including the evaluation value indicator 74 itself.

In a case where the pointer 64 in the memory map seek bar 70 is slid in the right direction so that the frame number indicated by the pointer 64 becomes n, the evaluation value indicator 74 moves to the leftmost position of the screening graph 72, as shown in FIG. 6C. Then, the evaluation value of the frame number n is shown at a left end of the screening graph 72. In a case where the pointer 64 is further slid in the right direction, as shown in FIG. 6D, the evaluation value indicator 74 is also moved in the right direction, and the respective evaluation values corresponding to the B-mode image data of the frame numbers n to n+j are displayed in an overwritten manner on the left side of the evaluation value indicator 74, including the evaluation value indicator 74 itself. A state in which the respective evaluation values corresponding to the B-mode image data of the frame numbers j+1 to n−1 are displayed is maintained on the right side of the evaluation value indicator 74, excluding the evaluation value indicator 74 itself.

In a case where the pointer 64 in the memory map seek bar 70 is slid to the right end again so that the pointer 64 reaches the frame number 2n, the evaluation value indicator 74 moves to the left end of the screening graph 72 as shown in FIG. 6E, and the evaluation value of the frame number 2n is shown on the leftmost side of the screening graph 72. In a case where the pointer 64 is further slid in the right direction, the evaluation value indicator 74 is also moved in the right direction, and the respective evaluation values corresponding to the B-mode image data of the frame numbers 2n to 2n+j are displayed in an overwritten manner on the left side of the evaluation value indicator 74, including the evaluation value indicator 74 itself. A state in which the respective evaluation values corresponding to the B-mode image data of the frame numbers n+j+1 to 2n−1 are displayed is maintained on the right side of the evaluation value indicator 74, excluding the evaluation value indicator 74 itself.

In this way, in a case where the pointer 64 in the memory map seek bar 70 is moved in the right direction, the evaluation value indicator 74 of the screening graph 72 is also moved in the right direction. Each time the pointer 64 is moved in the right direction and the frame number reaches a multiple of n, the evaluation value indicator 74 returns discontinuously to the left end. In a case where the evaluation value indicator 74 is at an intermediate position on the frame number axis of the screening graph 72, a new evaluation value after the evaluation value indicator 74 returns discontinuously to the left end is displayed in an overwritten manner on the left side of the evaluation value indicator 74, including the evaluation value indicator 74 itself. The display of the evaluation value before the evaluation value indicator 74 returns discontinuously to the left end is maintained on the right side of the evaluation value indicator 74, excluding the evaluation value indicator 74 itself.

Here, the display in a case where the pointer 64 of the memory map seek bar 70 is moved in the right direction is shown. In a case where the pointer 64 is moved in the left direction, that is, in a case of the rewind playback, the rod-like elements 76 corresponding to frame numbers sequentially designated with the movement of the pointer 64 are displayed in a sequential arrangement from left to right. That is, the rod-like elements 76 corresponding to the frame numbers sequentially designated by the pointer 64 are displayed in a sequential arrangement from left to right, regardless of the direction in which the pointer 64 moves. The evaluation value indicator 74 moves from left to right together with the display of the latest rod-like element 76 regardless of the direction in which the pointer 64 moves, and discontinuously returns to the left end each time it reaches the right end.

The process executed by the ultrasound diagnostic apparatus 100 for the display of the screening graph 72 will be described below with reference to FIG. 1. The feature analysis unit 32 obtains an evaluation value for each item of the B-mode image data of the N frames stored in the cine memory 30. The evaluation value may be, for example, a category of a tumor appearing in the B-mode image (hereinafter, simply referred to as a category), a degree of ease of recognition of a boundary of the tumor appearing in the B-mode image (hereinafter, referred to as a boundary clarity), an aspect ratio of the tumor appearing in the B-mode image, or a level of reflected ultrasound. The level of the reflected ultrasound includes a level of ultrasound directly reflected by the tumor, a level of a posterior echo, and the like. The feature analysis unit 32 may store the evaluation value itself or may store the evaluation value in the storage unit 40 via the controller 42.

The feature analysis unit 32 outputs the evaluation value corresponding to the frame number designated by the pointer 64 in the memory map seek bar 70 to the reference image generation unit 34. The reference image generation unit 34 generates screening image data indicating the screening graph 72 based on the frame number designated by the pointer 64 in the memory map seek bar 70 and the evaluation value output from the feature analysis unit 32, and outputs the screening image data to the image combining unit 24. In addition, the reference image generation unit 34 generates seek bar image data indicating the memory map seek bar 70, and outputs the seek bar image data to the image combining unit 24.

The image combining unit 24 reads the B-mode image data corresponding to the frame number designated by the pointer 64 in the memory map seek bar 70 from the cine memory 30. The image combining unit 24 generates display image data indicating the B-mode image 54, the memory map seek bar 70, and the screening graph 72 based on the B-mode image data, and on the seek bar image data and the screening image data output from the reference image generation unit 34, and outputs the display image data to the display processing unit 26. The display processing unit 26 generates a video signal based on the display image data, and outputs the video signal to the display 46. The display 46 displays a display image including the B-mode image 54, the memory map seek bar 70, and the screening graph 72 based on the video signal.

As described above, in the ultrasound diagnostic apparatus 100, the evaluation values for n frames of the B-mode image data out of the B-mode image data of the N frames stored in the cine memory 30 are displayed on the screening graph 72. In this display, it may be difficult for the user to ascertain which B-mode image data of the N frames of B-mode image data stored in the cine memory 30 the evaluation value displayed on the screening graph 72 corresponds to.

Therefore, the reference image generation unit 34 according to the present embodiment generates seek bar image data for displaying the memory map seek bar 70 as shown in FIG. 4 instead of the seek bar 60 shown in FIG. 3, and outputs the seek bar image data to the image combining unit 24. The memory map seek bar 70 is obtained by superimposing a memory map indicating a range of the frame numbers targeted for display on the screening graph 72 on the seek bar 60 shown in FIG. 3. In addition to the B-mode image 54 and the screening graph 72, a display image including the memory map seek bar 70 is displayed on the display 46.

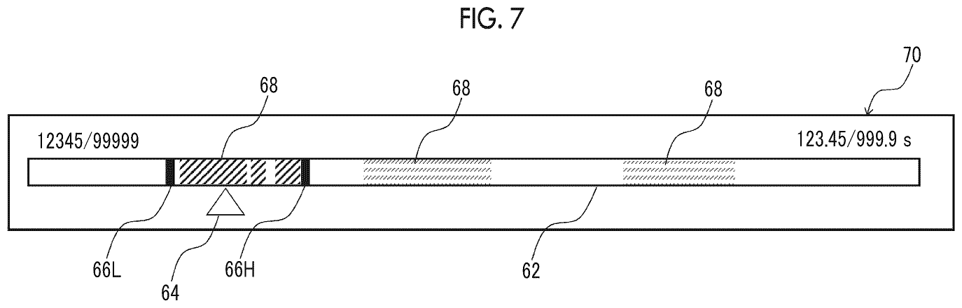

FIG. 7 shows an example of the memory map seek bar 70. In the memory map seek bar 70, a lower limit scale 66L and an upper limit scale 66H are marked on the band-shaped FIG. 62. The lower limit scale 66L indicates the minimum value of the frame numbers in which the evaluation value is indicated on the screening graph 72, and the upper limit scale 66H indicates the maximum value of the frame numbers in which the evaluation value is indicated on the screening graph 72. A range from the frame number indicated by the lower limit scale 66L to the frame number indicated by the upper limit scale 66H is an evaluation range in which the evaluation value is indicated on the screening graph 72. As described above, the lower limit scale 66L and the upper limit scale 66H are figures that display, on the memory map, the evaluation range on the frame number axis targeted for display on the screening graph 72.

In the band-shaped FIG. 62, an attention region 68 where the evaluation value exceeds the threshold value Th is shown in a range corresponding to the evaluation range of the memory map seek bar 70. The attention region 68 may be shown differently from other regions in the band-shaped FIG. 62. For example, the attention region 68 may be colored differently from the other regions, or may be filled with a pattern different from the other regions. The display of the attention region 68 indicates that the evaluation value for the B-mode image data corresponding to the frame number belonging to the attention region 68 exceeds the threshold value Th. As shown in FIG. 7, in the memory map seek bar 70, different fill patterns may be applied to the attention region 68 within the evaluation range and the attention region 68 outside the evaluation range.

As described above, in the ultrasound diagnostic apparatus 100 according to the present embodiment, a range of the frame numbers of the ultrasound image data whose evaluation value satisfies a lesion condition among the B-mode image data of a plurality of frames stored in the cine memory 30 is determined. Here, the lesion condition is a condition in which the evaluation value exceeds the threshold value Th. Then, the range of the frame numbers of the ultrasound image data whose evaluation value exceeds the threshold value Th is displayed as the attention region 68 on the memory map seek bar 70.

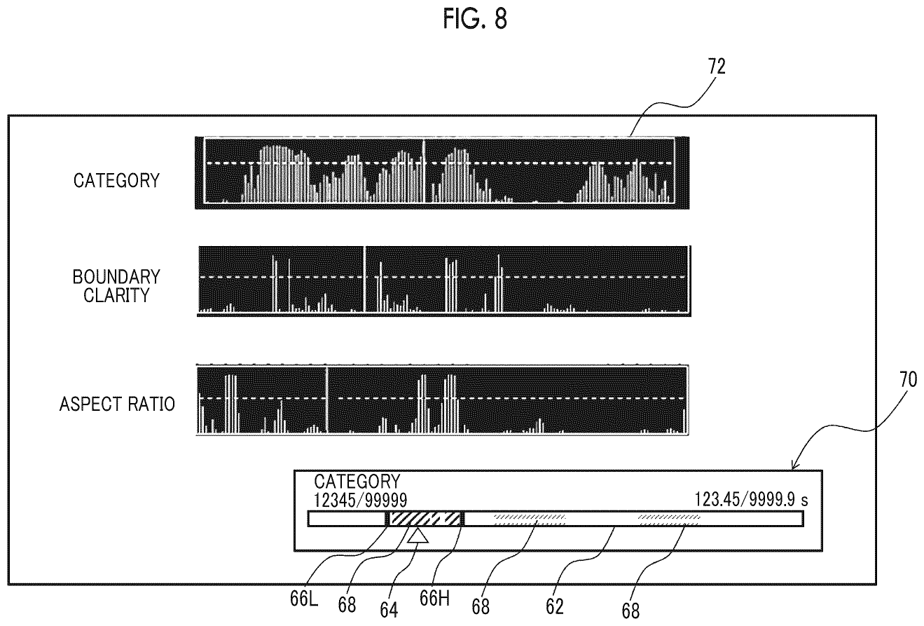

The display 46 may display the screening graph 72 for each of a plurality of different types of evaluation values. FIG. 8 shows an example in which screening graphs 72 for the category, the boundary clarity, and the aspect ratio are arranged and displayed in the vertical direction on the display 46. Although not shown in the drawing, these screening graphs 72 may be displayed on the display 46 together with the B-mode image designated by the memory map seek bar 70.

In a case where the screening graph 72 is displayed for each of a plurality of different types of evaluation values, one of a plurality of the screening graphs 72 in which the lower limit scale 66L, the upper limit scale 66H, and the attention region 68 (hereinafter, referred to as a memory map element) are displayed by the memory map seek bar 70 may be selected by the operation on the operation unit 44. In addition, the type of the evaluation value (the evaluation value to be displayed on the memory map) of the screening graph 72 whose memory map is displayed on the memory map seek bar 70 may be displayed on the memory map seek bar 70. In the example shown in FIG. 8, the evaluation value of the memory map display target is indicated as a category.

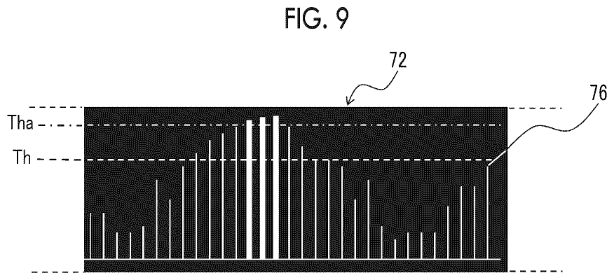

On the screening graph 72, the rod-like element 76 with an evaluation value larger than an attention threshold value Tha may be displayed in a manner different from the rod-like element 76 with an evaluation value equal to or less than the attention threshold value Tha. Here, the attention threshold value Tha is a value larger than the threshold value Th. For example, the rod-like element 76 with an evaluation value greater than the attention threshold value Tha may be displayed with a color or a thickness different from that of the rod-like element 76 with an evaluation value equal to or less than the attention threshold value Tha. In addition, brightness of the rod-like element 76 with an evaluation value greater than the attention threshold value Tha may be made greater than brightness of the rod-like element 76 with an evaluation value equal to or less than the attention threshold value Tha. FIG. 9 shows a part of such a screening graph 72. In the example shown in FIG. 9, the rod-like element 76 with an evaluation value greater than the attention threshold value Tha is displayed with a thickness different from the rod-like element 76 with an evaluation value equal to or less than the attention threshold value Tha. The rod-like element 76 with an evaluation value greater than the attention threshold value Tha may be displayed with brightness or a color different from that of the rod-like element 76 with an evaluation value equal to or less than the attention threshold value Tha.

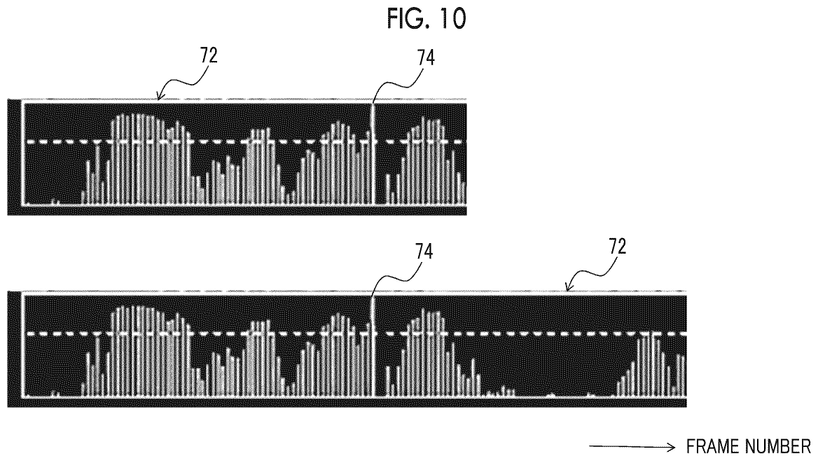

The number of frames or a time length of the frames to be displayed on the screening graph 72 may be changed by the operation on the operation unit 44. In an upper part and a lower part of FIG. 10, screening graphs 72 having different numbers of frames are shown for comparison. The number of frames of the screening graph 72 shown in the lower part is 1.5 times the number of frames of the screening graph 72 shown in the upper part. One of the screening graph 72 in the upper part and the screening graph 72 in the lower part may be displayed on the display 46. In a case where the B-mode image data is acquired at a frame rate of 100 frames per second and the screening graph 72 in the upper part is a screening graph 72 with a time length of 10 seconds, the screening graph 72 in the lower part is a screening graph 72 with a time length of 15 seconds.

The ultrasound diagnostic apparatus 100 according to the present embodiment may have a function of extracting B-mode image data in which a lesion is likely to appear from the B-mode image data of a plurality of frames stored in the cine memory 30 and displaying an image based on the B-mode image data. This function may be realized by the technique disclosed in JP2021-108842A.

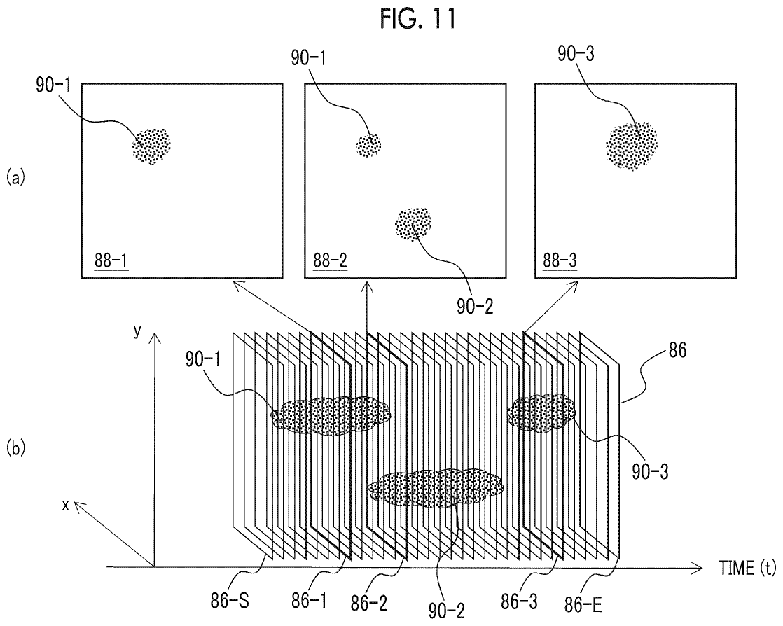

In a lower part of FIG. 11, the B-mode image data of a plurality of frames stored in the cine memory 30 and lesion candidate regions 90-1 to 90-3 are conceptually shown. The lesion candidate regions 90-1 to 90-3 correspond to regions of a tissue in the subject 50 that are likely to be lesions. That is, the lesion candidate regions 90-1 to 90-3 are atypical regions in which pixel values of pixels constituting the B-mode image are different from an average pixel value of surroundings pixels. The lesion candidate regions 90-1 to 90-3 may be defined by a region recognition process of recognizing a feature region in the B-mode image, as will be described below.

In the lower part of FIG. 11, the B-mode image data stored in the cine memory 30 is conceptually shown by a planar B-mode image. Each B-mode image 86 is acquired in a case where the ultrasound probe 12 is linearly moved over the subject 50 at a constant speed during the operation in the real-time display mode. An axis extending in the horizontal direction is a time axis (t-axis), and an xy plane is defined perpendicular to the time axis. A plane parallel to the xy plane is scanned with an ultrasonic beam, so that each B-mode image spreads parallel to the xy plane, and a plurality of the B-mode images 86 are linked together along the time axis. A B-mode image 86-S on the leftmost side corresponds to the earliest B-mode image data stored in the cine memory 30, and a B-mode image 86-E on the rightmost side corresponds to the last B-mode image data stored in the cine memory 30.

In an upper part of FIG. 11, each B-mode image that can be displayed on the display 46 in the freeze mode is schematically shown. In a case where the operation of the ultrasound diagnostic apparatus 100 is set to the freeze mode, an image based on the last B-mode image 86-E stored in the cine memory 30 is displayed on the display 46. In a case where a B-mode image 86-1 is designated by the operation of the memory map seek bar 70, a B-mode image 88-1 is displayed on the display 46. Similarly, in a case where a B-mode image 86-2 or 86-3 is designated, a B-mode image 88-2 or 88-3 is displayed on the display 46.

The feature analysis unit 32 shown in FIG. 1 executes a region recognition process of recognizing a feature region in the B-mode image on each B-mode image data, and obtains lesion candidate region data indicating a lesion candidate region on each B-mode image.

The feature analysis unit 32 includes the above-described evaluation value in the lesion candidate region data obtained for each B-mode image. The feature analysis unit 32 stores the lesion candidate region data obtained for each B-mode image in the storage unit 40 via the controller 42.

The feature analysis unit 32 may define the lesion candidate region from the B-mode image through the following binarization process. The feature analysis unit 32 executes a binarization process of setting a pixel value of a region where a pixel value exceeds a predetermined binarization threshold value to 1 and setting a pixel value of a region where a pixel value is equal to or less than the binarization threshold value to 0, and defines the region where the pixel value is 0 as a result of the binarization process as the lesion candidate region.

The feature analysis unit 32 may define the lesion candidate region through the following pattern matching. The feature analysis unit 32 reads reference data indicating patterns of a plurality of types of lesion candidate regions having different pixel values, sizes, shapes, and the like from the storage unit 40. The feature analysis unit 32 acquires the reference data and obtains a degree of similarity between each of the patterns of the plurality of types of lesion candidate regions and the B-mode image. The degree of similarity may be a correlation value obtained by a correlation calculation between an image showing a pattern of the lesion candidate region and the B-mode image. The feature analysis unit 32 defines the lesion candidate region in the B-mode image based on a pattern in which the correlation value exceeds a predetermined value.

The feature analysis unit 32 may define the lesion candidate region through the following region division. The region division is a process of extracting a region having predetermined features such as a shape, a size, and a pixel value from the B-mode image. The feature analysis unit 32 reads the reference data necessary for the region division from the storage unit 40. The feature analysis unit 32 defines the lesion candidate region in the B-mode image through the region division based on the reference data.

The feature analysis unit 32 obtains a lesion candidate frame number range that is a range of frame numbers in which the lesion candidate region appears. A frame number at which the evaluation value is maximized in the lesion candidate frame number range is obtained as an index (representative) frame number. In the example shown in FIG. 11, the feature analysis unit 32 obtains a lesion candidate frame number range for the lesion candidate regions 90-1 to 90-3, and obtains an index frame number for each of the lesion candidate regions 90-1 to 90-3. The obtained index frame numbers are frame numbers for the B-mode images 86-1 to 86-3.

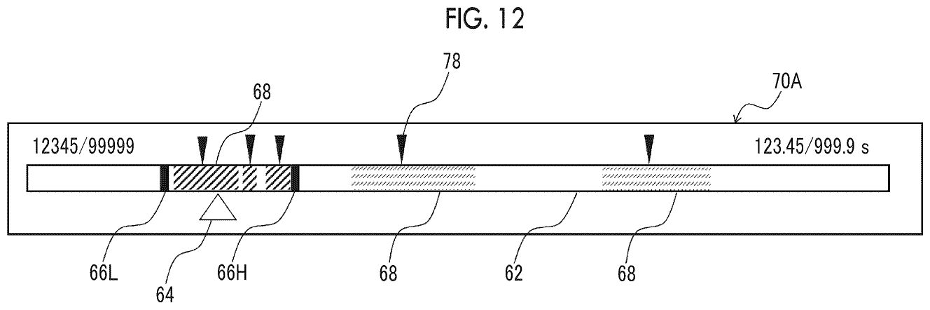

FIG. 12 shows a memory map seek bar 70A with an index frame point 78. The index frame point 78 indicates a position on a frame number axis corresponding to the index frame number.

As described above, B-mode image data whose evaluation value satisfies an index condition among the ultrasound image data of a plurality of frames stored in the cine memory 30 is determined as index ultrasound image data. Here, the index condition is a condition in which the evaluation value is maximized in the lesion candidate frame number range. The index frame point 78 indicating a storage location of the index ultrasound image data in the cine memory 30 with a frame number is displayed together with the memory map.

In a case where the memory map seek bar 70A shown in FIG. 12 is displayed, the B-mode image corresponding to the index frame number is displayed on the display 46 by matching the pointer 64 with the position of the index frame point 78. As a result, in a case where there are a plurality of lesion candidate regions in the subject 50, it is easy to perform an operation of displaying the B-mode image of the frame number at which the evaluation value is maximized in each lesion candidate region.

The present disclosure can also be applied to a program and a program product. An ultrasound image processing program according to the embodiment of the present disclosure causes a processor to execute a process comprising: acquiring an evaluation value for each item of ultrasound image data sequentially acquired based on transmission and reception of ultrasound; sequentially updating and displaying an evaluation graph showing the evaluation value in a case where ultrasound images based on the ultrasound image data sequentially acquired are displayed sequentially; and displaying a memory map for the ultrasound image data sequentially acquired based on the transmission and the reception of the ultrasound. The ultrasound image processing program may be provided by being stored in a storage medium such as a memory card, a USB memory, or a CD-ROM.

What is claimed is:

1. An ultrasound image processing apparatus comprising:
a processor configured to
acquire an evaluation value for each item of ultrasound image data sequentially acquired based on transmission and reception of ultrasound,
sequentially update and display an evaluation graph showing the evaluation value in a case where ultrasound images based on the ultrasound image data sequentially acquired are displayed sequentially, and
display a memory map for the ultrasound image data sequentially acquired based on the transmission and the reception of the ultrasound,
wherein the memory map indicates a storage location in a memory where each item of the ultrasound image data is stored.

2. The ultrasound image processing apparatus according to claim 1,
wherein the memory map is a map that indicates the storage location in the memory with a frame number,
the evaluation graph is a graph in which the evaluation value is displayed in association with a frame number axis, and
the processor is configured to display, on the memory map, an evaluation range on the frame number axis that is targeted for display in the evaluation graph in a form of a figure.

3. The ultrasound image processing apparatus according to claim 2,
wherein the processor is configured to determine a range of frame numbers of ultrasound image data whose evaluation value satisfies a lesion condition among the ultrasound image data of a plurality of frames stored in the memory, and to display the determined range on the memory map.

4. The ultrasound image processing apparatus according to claim 2,
wherein the processor is configured to
determine index ultrasound image data whose evaluation value satisfies an index condition among the ultrasound image data of a plurality of frames stored in the memory, and
display an index frame point indicating a storage location in the memory where the index ultrasound image data is stored, together with the memory map.

5. The ultrasound image processing apparatus according to claim 1,
wherein the processor is configured to display a memory map operation figure obtained by superimposing the memory map on an operation figure for designating any of the ultrasound image data of a plurality of frames stored in the memory, and to display an image based on the ultrasound image data designated by the operation figure.

6. The ultrasound image processing apparatus according to claim 1,
wherein the evaluation graph is a graph in which elongated figures that extend in a first axis direction with lengths corresponding to the evaluation values are displayed in a sequential arrangement in a second axis direction as the ultrasound images based on the ultrasound image data are sequentially displayed.

7. The ultrasound image processing apparatus according to claim 1,
wherein the evaluation value is a value indicating a degree to which the ultrasound image indicated by the ultrasound image data includes an atypical region.

8. The ultrasound image processing apparatus according to claim 1,
wherein the processor is configured to arrange and display a plurality of the evaluation graphs indicating a plurality of different types of the evaluation values.

9. The ultrasound image processing apparatus according to claim 8,
wherein the different types of the evaluation values are at least two values of a tumor category, a degree of ease of recognition of a tumor boundary, a tumor aspect ratio, and a level of reflected ultrasound.

10. A storage medium storing an ultrasound image processing program for causing a processor to execute a process comprising:
acquiring an evaluation value for each item of ultrasound image data sequentially acquired based on transmission and reception of ultrasound;
sequentially updating and displaying an evaluation graph showing the evaluation value in a case where ultrasound images based on the ultrasound image data sequentially acquired are displayed sequentially; and
displaying a memory map for the ultrasound image data sequentially acquired based on the transmission and the reception of the ultrasound.
